SEQUENCE LISTING (10 sequences)

SEQ ID NO: 1 - DNA, 38 bp, Escherichia coli
acacgtgctcagcgattgtgtaggctggagctgcttcg

SEQ ID NO: 2 - DNA, 39 bp, Escherichia coli
gccatgctcgagatgaatatcctccttagttcctattcc

SEQ ID NO: 3 - DNA, 36 bp, Bacillus subtilis
attacgcatatgtttggctaacacacacgccattcc

SEQ ID NO: 4 - DNA, 36 bp, Bacillus subtilis
ctttttcgatcgtttccgacgcttattcgcttcgct

SEQ ID NO: 5 - DNA, 31 bp, Clostridium perfringens
gtttgatcatggtctttgtactaacctgtgg

SEQ ID NO: 6 - DNA, 22 bp, Clostridium perfringens
tacagcatgaccgttaaagtgg

SEQ ID NO: 7 - DNA, 50 bp, Clostridium perfringens
ccactttaacggtcatgctgtaagtgcaaggtacacttgcaaagtagtgg

SEQ ID NO: 8 - DNA, 32 bp, Clostridium perfringens
ggatcgatccaacttaatcgccttgcagcaca

SEQ ID NO: 9 - DNA, 23 bp, Clostridium perfringens
gggaggtcaatctatgaaatgcg

SEQ ID NO: 10 - DNA, 24 bp, Clostridium perfringens
cggagcatttggctttccttccat

(19) United States
(12) Patent Application Publication
(10) Pub. No.: US 2014/0193916 A1
(43) Pub. Date: Jul. 10, 2014

(54) Electrotransformation of Clostridium pasteurianum

(71) Applicants: Michael Evan Pyne, Hamilton (CA); Murray Moo Young, Waterloo (CA); Duane Andrew Chung, Mississauga (CA); Chih-Hsiung Perry Chou, Waterloo (CA)

(72) Inventors: Michael Evan Pyne, Hamilton (CA); Murray Moo Young, Waterloo (CA); Duane Andrew Chung, Mississauga (CA); Chih-Hsiung Perry Chou, Waterloo (CA)

(73) Assignee: Algaeneers Inc., Hamilton (CA)

(21) Appl. No.: 14/150,764

(22) Filed: Jan. 8, 2014

Related U.S. Application Data
(60) Provisional application No. 61/750,335, filed on Jan. 8, 2013.

(51) Int. Cl. C12N 15/74

(52) U.S. Cl. CPC C12N 15/74
USPC 435/471; 435/252.3

(57) ABSTRACT

By this invention, for the first time, a method for high-efficiency genetic transformation of the anaerobic bacterium Clostridium pasteurianum is provided. Clostridium pasteurianum is a bacterium of substantial industrial importance, due to its selectivity and high productivity of the biofuel and biochemical n-butanol, and its ability to grow on a wide variety of inexpensive substrates. Notable among the substrates that it can utilize as a sole source of carbon and energy is glycerine, which is produced in increasing quantities globally as a by-product of biodiesel processing. The industrial exploitation of Clostridium pasteurianum has previously been impeded by the lack of genetic engineering tools for this bacterium. This invention provides such tools for the first time. Included in the invention is a means for protecting newly introduced DNA from degradation by a restriction enzyme within C. pasteurianum. Then, a detailed protocol is given, which enables high-efficiency transformation of C. pasteurianum via a series of treatments and electroporation conditions which successfully negotiate the resistant cell wall of C. pasteurianum. Finally, the invention discloses selection markers and vector components, which round out the tools required to successfully perform genetic engineering in C. pasteurianum for the first time.

### TECHNICAL FIELD

The present invention is directed to bacterial cells and methods for introducing nucleic acids into bacterial cells, and methods and nucleic acids related thereto.

### BACKGROUND

Biofuels are regarded as offering a sustainable and environmentally positive replacement for some fossil fuels. Typically, the problem with biofuels is not their performance, but their cost. Most biofuels, in the absence of government subsidies, are more expensive than their fossil fuel counterparts. Thus, reducing the cost of biofuels remains one of the biggest priorities in the biofuel industry. In an analysis of biofuel costs, the cost of the raw feedstock from which the biofuels is produced, is normally the major cost item. For example, in ethanol fuel produced by yeast fermentation, the cost of the sugar consumed by the yeast in the fermentation process represents the major production cost. As a result, biofuel producers looking to reduce their production costs are exploring the use of alternative low-cost feedstocks.

One biofuel-producing microorganism, which is particularly attractive in terms of both its ability to utilize a variety of inexpensive feedstocks and also to produce relatively large amounts of n-butanol, a very attractive biofuel that is much more similar to gasoline than is ethanol, is C. pasteurianum. C. pasteurianum is often found in whey isolates and it has a strong ability to utilize waste sugars generated during dairy processing. However, perhaps the most important inexpensive feedstock that C. pasteurianum utilizes very well is glycerine. Glycerine is produced as a major (i.e. 10%) by-product in biodiesel processing. At present, C. pasteurianum is the only known bacterium that can grow directly on glycerine as its sole carbon and energy source, while producing butanol as its major metabolite and product.

Molecular biology, and genetic engineering in particular, has, in some cases, emerged as a powerful approach for improving the biofuel production capacity, as well as feedstock utilization range, of industrial microorganisms. Today, there several major companies, such as Gevo, Butamax, and Cobalt Biofuels, that use genetically engineered organisms to produce biobutanol (that is, butanol produced from a biological process, as opposed to butanol produced by a chemical processing that does not involve living organisms) from a fermentation process. However, the ability to perform genetic engineering in a microorganism requires some basic tools such as: the ability to introduce new DNA into the microorganism, the ability to select for microorganisms carrying the new DNA, the ability to maintain the new DNA within the microorganism in a functional form (i.e. in a form that resists host defense and DNA degradation mechanisms), and the ability to express foreign genes within the microorganism. Prior to the present invention, such tools were lacking for C. pasteurianum; therefore C. pasteurianum was not amenable to genetic engineering and it has not previously been genetically engineered. In fact, out of the many species of Clostridial bacteria that have been found, only a very small number of them (approximately 5) have been developed for genetic engineering.

This invention discloses for the first time, the first successful genetic engineering of C. pasteurianum. This invention, together with the recently published genome of C. pasteurianum, provides the tools that will enable C. pasteurianum to be fully harnessed and developed for industrial butanol and biofuel production for the first time ever. Moreover, the tools that were developed to transform C. pasteurianum with foreign DNA will have broad applicability to enabling the transformation of bacteria that have not previously been genetically transformed.

### SUMMARY OF THE INVENTION

The present invention provides protocols that enable recombinant DNA constructs to be introduced into bacterial cells for which such protocols have not previously been disclosed. The present invention includes the bacterial cells containing recombinant DNA constructs.

In one preferred embodiment, the bacteria cells are of the anaerobic bacterium Clostridium pasteurianum. In another preferred embodiment, the invention describes one or more methyltransferases and their means of application, which are required to pretreat recombinant DNA constructs in order to enable their successful introduction into said bacterial cells. In another preferred embodiment, means and conditions are disclosed whereby said bacterial cells are rendered more amenable to transformation by recombinant DNA constructs by the application of one or more electrical pulses delivered to the bacterial cells while in the presence of the recombinant DNA constructs. In another preferred embodiments, information regarding antibiotic selection markers and recombinant DNA origins of replication is disclosed whereby one skilled in the art would be enabled to construct recombinant DNA constructs which can persist in the bacterial cells following transformation as independent genetic entities termed plasmids or which could enable such recombinant DNA constructs to become integrated into the bacterial genome under antibiotic selection pressure.

### DESCRIPTION OF THE FIGURES

FIG. 1. M.FnuDII methyltransferase-mediated protection of pMTL85141 against CpaAI endonuclease. A. Time course digestion of pMTL85141 using crude protoplast extracts possessing CpaAI restriction activity, resolved on a 2% agarose gel. Digestion reactions contained 1.0 μg pMTL85141 and 25% protoplast extract in a total volume of 1×CpaAI custom buffer. For comparison, pMTL85141 is shown undigested and digested with BstUI, a commercial isoschizomer of CpaAI. Expected digestion products are 1785, 581, 270, 252, and 75 bp. B. M.FnuDII-mediated protection of pMTL85141 from CpaAI digestion (left panel). Protoplast extract digestions contained 1.0 μg pMTL85141 or pMTL85141+pFnuDIIMKn, the vector harboring the M.FnuDII methyltransferase gene, and 25% protoplast extract in a total volume of 1×CpaAI custom buffer. pMTL85141 preparation in the presence of plasmid pFnuDIIMKn afforded protection of pMTL85141 from both BstUI and CpaAI restriction, as no digestion products could be detected. Methylation treatment resulted in the presence of high-molecular weight bands. Linearization of the high molecular weight bands by NcoI digestion (right panel) confirmed the presence of pMTL85141 (2,963 bp) and the methylating plasmid, pFnuDIIMKn (6,449 bp), at the correct sizes of the individual linearized vectors.

FIG. 2. Low-level electrotransformation of C. pasteurianum. A. Colony PCR confirmation of pMTL85141 presence in C. pasteurianum transformants using primers pMTL.seq.S (SEQ ID NO: 9) and pMTL.seq.AS (SEQ ID NO: 10). The expected product of 518 bp could only be amplified from E. coli transformed with pMTL85141 and from thiamphenicol-resistant C. pasteurianum colonies, and not from non-recombinant (Zhao, et al., 2003) C. pasteurianum. B. XhoI-linearized pMTL85141 plasmid prepared from E. coli DH5α and from a representative transformant of C. pasteurianum showing the expected plasmid size of 2,963 bp. Some undigested vector remains visible in the E. coli preparation.

FIG. 3. Investigation of cell-wall-weakening and osmoprotection on electrotransformation of C. pasteurianum. A. Investigation of cell-wall-weakening agents. Cells were grown to early exponential phase (OD₆₀₀ 0.3-0.4) and glycine (gly) or DL-threonine (thr) was added along with 0.25 M sucrose (suc). For lysozyme and penicillin G treatments, additives were supplemented to buffer SMP prior to electroporation and incubated anaerobically at 37° C. for 30 minutes. An untreated culture was included as a control. The OD₆₀₀ of each culture at time of harvest is shown (O). Pulse duration was unaffected between samples. B. Investigation of glycine and sucrose concentrations. Six cultures were grown to an OD₆₀₀ of 0.4 and glycine was added to a final concentration of 0.75, 1.0, or 1.25% together with sucrose at 0.25 (light shading) or 0.4 M (dark shading). Growth was minimally affected between samples, as all cultures attained a final OD₆₀₀ of 1.2-1.5. Pulse duration was unaffected between samples. C. Investigation of glycine concentration and duration of exposure. Two cultures were grown to an OD₆₀₀ of 0.4 and glycine was added to a final concentration of 0.75 or 1.25% together with 0.4 M sucrose. An additional control culture was prepared without either glycine or sucrose supplementation. Cells were harvested, washed, and electroporated at either 2.5 (light shading) or 4 hours (dark shading) following supplementation with glycine and sucrose. Pulse duration was unaffected between samples. D. Effect of sucrose concentration within the wash and electroporation buffer. Cultures were washed and electroporated in SMP buffer containing either isotonic (0.27 M) or hypertonic sucrose (0.5 M). Pulse duration was unaffected between samples. E. Effect of sucrose concentration within the outgrowth medium. Cultures were electroporated and resuspended and grown in 2xYTG medium containing either 0.2 or 0.4 M sucrose.

FIG. 4. Investigation of membrane permeabilization on the electrotransformation of C. pasteurianum. Immediately prior to pulse delivery, cell-DNA suspensions were supplemented with 5, 10, or 15% ethanol (EtOH) or 1 or 2% butanol (BuOH). An untreated sample was included as a control. The time constant of each pulse is shown (◯). The sample treated with 2% butanol failed to grow during the allotted 16-hour recovery period following electroporation.

FIG. 5. Investigation of electric pulse parameters on the electrotransformation of C. pasteurianum. A. Effect of pulse voltage (field strength). Electrotransformation efficiency was measured using electric pulses of 1.6, 1.8, or 2.0 kV, corresponding to field strengths of 4.0, 4.5, and 5.0 kV cm⁻¹. The time constant of each pulse is shown (◯). B. Effect of pulse capacitance. Electrotransformation efficiency was measured at 25 (light shading) and 50 μF (dark shading) under voltages of 1.8 and 2.25 kV. The time constant of each pulse is shown (◯). C. Effect of pulse resistance. Electrotransformation efficiency was measured at 200, 600, and ∞Ω at a voltage of 2.25 kV. The time constant of each pulse is shown (◯).

FIG. 6. Investigation of amount of DNA and outgrowth duration on the electrotransformation of C. pasteurianum. A. Effect of plasmid DNA amount on total number of transformants and electrotransformation efficiency of C. pasteurianum. Separately, 0, 0.25, 0.5, 1.0, 2.5, and 5.0 μg of pMTL85141 were added to electrocompetent cells of C. pasteurianum and electroporated. Total number of thiamphenicol-resistant transformants (light shading) and electrotransformation efficiency (dark shading) were quantified. Pulse duration was unaffected between samples. Zero μg pMTL85141 failed to generate thiamphenicol-resistant transformants. B. Effect of post-electroporation incubation time. Cells were electroporated, transferred to 10 ml outgrowth medium containing 0.2 M sucrose, and incubated for 0, 2, 4, 6, or 16 hours prior to selective plating.

### DEFINITIONS

“Gene” refers to a nucleic acid sequence that encompasses a 5′ promoter region associated with the expression of the gene product, any intron and exon regions and 3′ or 5′ untranslated regions associated with the expression of the gene product.

“Transgene” refers to a nucleic acid sequence associated with the expression of a gene introduced into an organism. A transgene includes, but is not limited to, an endogenous gene or a gene not naturally occurring in the organism. A “transgenic organism” is any organism that stably incorporates a transgene in a manner that facilitates transmission of that transgene from the organism by any sexual or asexual method.

### Tables

TABLE 1
Strains, plasmids, and oligonucleotides.

| Strain | Relevant characteristics | Source or reference |
|---|---|---|
| Escherichia | F⁻ endA1 glnV44 thi-1 recA1 relA1 gyrA96 deoR nupG φ80dlacZΔM15 | Lab stock |
| coli DH5α | Δ(lacZYA-argF)U169, hsdR17(r_{K}⁻m_{K}⁺), λ⁻ | |
| Escherichia | F⁻ endA1 glnV44 thi-1 relA1? e14⁻(mcrA⁻) rfbD1? spoT1? Δ(mcrC- | Lab stock; New |
| coli ER1821 | mrr)114::IS10 | England Biolabs |
| Clostridium | Wild-type | American Type Culture |
| pasteurianum | | Collection |
| ATCC 6013 | | |
| Plasmid | Relevant characteristics | Source or reference |
| pET-20b(+) | E. coli pET-series expression vector (Ap^{R}; ColE1 ori) | Novagen |
| pETKnFRT | Derived by inserting the FRT-flanked kan gene of pKD4 into the MCS of | This study |
| | pET-20b(+)(Ap^{R}; ColE1 ori; FRT-Kn^{R}-FRT) | |
| pFnuDIIM | The M.FnuDII methyltransferase gene of Fusobacterium nucleatum | (Lunnen, et al., 1988) |
| | inserted into the tet gene of pACYC184 (p15A ori; Cm^{R}) | |
| pFnuDIIMKn | Derived by inserting the FRT-flanked kan gene of pETKnFRT into | This study |
| | the cat gene of pFnuDIIM (p15A ori; Kn^{R}) | |
| pHT3 | E. coli-C. pasteurianum shuttle vector containing lacZ from | (Tummala, et al., |
| | Thermoanaerobacterium thermosulfurogenes EM1 (Ap^{R}; ColE1 ori; Erm^{R}; | 1999) |
| | pIM13 ori) | |
| pIMP1 | E. coli-C. pasteurianum shuttle vector (Ap^{R}; ColE1 ori; | (Mermelstein, et al., |
| | Erm^{R}; pIM13 ori) | 1992) |
| pKD4 | Template vector (Ap^{R}; pR6K ori; FRT-Kn^{R}-FRT) | (Datsenko and |
| | | Wanner, 2000) |
| pMTL82151 | E. coli-C. pasteurianum shuttle vector (Cm^{R}; ColE1 ori; pBP1 ori) | (Heap, et al., 2009) |
| pMTL83151 | E. coli-C. pasteurianum shuttle vector (Cm^{R}; ColE1 ori; pCB102 ori) | (Heap, et al., 2009) |
| pMTL84151 | E. coli-C. pasteurianum shuttle vector (Cm^{R}; ColE1 ori; pCD6 ori) | (Heap, et al., 2009) |
| pMTL85141 | E. coli-C. pasteurianum shuttle vector (Cm^{R}; ColE1 ori; pIM13 ori) | (Heap, et al., 2009) |
| pMTL85141ermB | Derived by insertion of the ermB gene of pIMP1 into pMTL85141 | This study |
| pSC12 | E. coli-C. pasteurianum shuttle vector (Cm^{R}; ColE1 ori; pIM13 ori) | (Zhao, et al., 2003) |
| pSY6 | E. coli-C. pasteurianum expression vector carrying the L. lactis | (Shao,et al., 2007) |
| | ItrB group II intron under control of the C. acetobutylicum | |
| | ptb promoter, and ItrA ORF(Ap^{R}; ColE1 ori; Erm^{R}; pIM13 ori) | |
| pSY6catP | Derived by replacing the ermB gene of pSY6 with the catP gene from | This study |
| | pSC12 | |
| Oligo- | | |
| nucleotide | Sequence (5'-3')* | SEQ ID |
| KnFRT.Blpl.S | ACACGTGCTCAGCGATTGTGTAGGCTGGAGCTGCTTCG | SEQ ID NO: 1 |
| KnFRT.XhoI.AS | GCCATGCTCGAGATGAATATCCTCCTTAGTTCCTATTCC | SEQ ID NO: 2 |
| ermB.NdeI.S | ATTACGCATATGTTTGGCTAACACACACGCCATTCC | SEQ ID NO: 3 |
| ermB.PvuI.AS | CTTTTTCGATCGTTTCCGACGCTTATTCGCTTCGCT | SEQ ID NO: 4 |
| catP.BcII.S | GTTTGATCATGGTCTTTGTACTAACCTGTGG | SEQ ID NO: 5 |
| pSC12.S0E.AS | tacagcatgaccgttaaagtgg | SEQ ID NO: 6 |
| pSC12.S0E.S | ccactttaacggtcatgctgtaAGTGCAAGGTACACTTGCAAAGTAGTGG | SEQ ID NO: 7 |
| catP.ClaI.AS | GGATCGATCCAACTTAATCGCCTTGCAGCACA | SEQ ID NO: 8 |
| pMTL.seq.S | GGGAGGTCAATCTATGAAATGCG | SEQ ID NO: 9 |
| pMTL.seq.AS | CGGAGCATTTGGCTTTCCTTCCAT | SEQ ID NO: 10 |

*Lower case: overlap sequences used in SOE PCR; Underline: restriction recognition sequences

TABLE 2
Consensus clostridial electrotransformation conditions leading to initial low-level transformation
of C. pasteurianum

| Electrotransformation | Consensus for Clostridium | Low-level transformation | |
|---|---|---|---|
| parameter | species | of C. pasteurianum | Selected references |
| Cell growth | | | |
| Growth medium | YTG or 2xYTG | 2xYTG | (Allen and Blaschek, |
| | | | 1988) |
| Growth phase and OD₆₀₀ | mid to late exponential phase | OD₆₀₀ 0.6-0.8 | (Tardif, et al., 2001) |
| at time of harvest | (OD₆₀₀ 0.5-0.9) | | |
| Washing and pulse delivery | | | |
| Wash and | 5-7 mM sodium phosphate, pH | 5 mM sodium phosphate, | (Mermelstein, et al., 1992, |
| electroporation buffer | 6.5-7.4, containing 270 mM | pH 6.5, containing 270 mM | Zhu, et al., 2005) |
| | sucrose and 1 mM MgCl₂ | sucrose and 1 mM MgCl₂ | |
| Number of wash steps | 1 | 1 | (Allen and Blaschek, |
| | | | 1988, Nakotte, et al., |
| | | | 1998) |
| Cuvette gap width | 0.4 cm | 0.4 cm | (Zhu, et al., 2005, |
| | | | Nakotte, et al., 1998, |
| | | | Zhou and Johnson, 1993) |
| Volume of cells | 600 μl | 600 μl | (Mermelstein, et al., |
| | | | 1992) |
| Pulse parameters | 2.0-2.5 kV; 25 μF; 200-800 Ω; | 2.0-2.5 kV; 25 μF; 200-800 Ω; | (Allen and Blaschek, |
| | 4-8 ms | 6-9 ms | 1988, Zhou and Johnson, |
| | | | 1993, Klapatch, et al., |
| | | | 1996) |
| Outgrowth | | | |
| Recovery and plating | YTG or 2xYTG | 2xYTG | |
| medium | | | |
| Transformation efficiency | Up to 10⁶ transformants μg⁻¹ | 2.4 × 10¹ transformants μg⁻¹ | (Tyurin, et al., 2000) |
| | DNA | pMTL85141 | |

TABLE 3
Summary of protocol for high-level electrotransformation of C. pasteurianum and
comparison to initial low-level protocol

| Electrotransformation | | |
|---|---|---|
| parameter | Low-level protocol | High-level protocol |
| Cell growth | | |
| Growth additive | None | 1.25% glycine (at OD₆₀₀ 0.3-0.4) |
| Osmotic stabilizer | None | Hypertonic sucrose (0.4M; at OD₆₀₀ |
| | | 0.3-0.4) |
| OD₆₀₀ at time of harvest | OD₆₀₀ 0.6-0.8 | OD₆₀₀ 0.6-0.8 |
| Washing and pulse delivery | | |
| Osmotic stabilizer | Isotonic sucrose (0.27M) | Isotonic sucrose (0.27M) |
| Cell membrane solubilizer | None | 5% (v/v) ethanol 5 min prior to pulse |
| DNA amount | 5 μg | 0.5 μg |
| Pulse parameters | 2.0-2.5 kV; 25 μF; 200-800 Ω; 6-9 ms | 1.8 kV; 25 μF; ∞ Ω; 12-14 ms |
| Outgrowth | | |
| Osmotic stabilizer | None | Hypotonic sucrose (0.2M) |
| Recovery time | 16 h | 4-6 h |
| Transformation efficiency | 2.4 × 10¹ transformants μg⁻¹ | Up to 7.5 × 10⁴ transformants μg⁻¹ |
| | pMTL85141 | pMTL85141 |

### DETAILED DESCRIPTION OF INVENTION

The invention consists of 2 parts:

1. Overcoming a CpaAI restriction enzyme within C. pasteurianum.

2. Overcoming the low electroporation transformation efficiency of C. pasteurianum.

A third important aspect of the invention is the development of DNA vectors and selection markers which enable the expression of foreign genes within C. pasteurianum.
1 Overcoming the CpaAI Restriction Enzyme within C. pasteurianum

Based on early genetic studies, it appears efforts were in place to conduct genetic manipulation of C. pasteurianum, since a method for producing and regenerating protoplasts (i.e. cells lacking cell walls) was developed (Clarke, et al., 1979) and a Type-II restriction endonuclease was identified as a potential barrier to gene transfer (Richards, et al., 1988). Successful conjugation-based plasmid transfer to C. pasteurianum has also been documented (Richards, et al., 1988), yet no protocol has been described, nor have any genetic mutants arisen from any prior work. Accordingly, no genetic tools are currently available for the manipulation of C. pasteurianum.

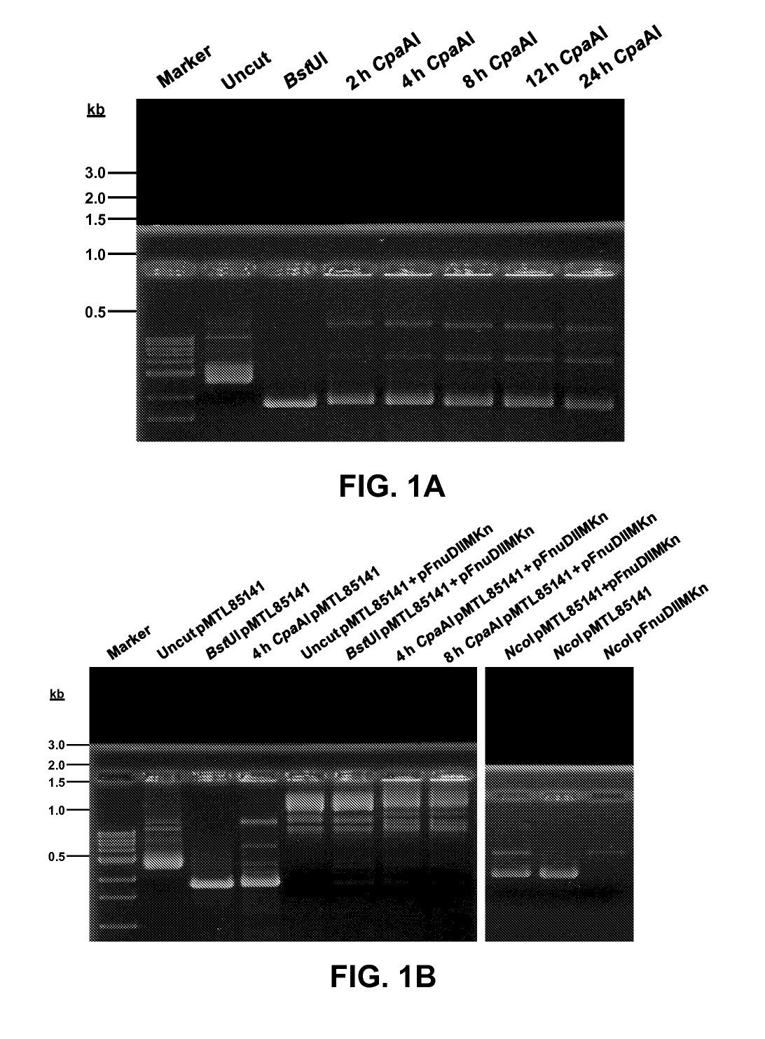

To develop a C. pasteurianum transformation protocol, we first assayed crude cell lysates for the presence of restriction-modification systems, which potently inhibit plasmid DNA transfer to bacteria. At least one Type-II restriction endonuclease, designated CpaAI with 5′-CGCG-3′ recognition and an isoschizomer of ThaI and FnuDII, has been previously identified in cell-free lysates of C. pasteurianum ATCC 6013 (Richards, et al., 1988). We initially prepared crude cell lysates through sonication of whole cells. As found in other species, such as C. acetobutylicum, lysates generated in this manner potently degraded all plasmid DNA substrates, presumably due to non-specific cell-wall-associated nucleases (data not shown). To overcome non-specific nuclease activity, we then aimed to assay CpaAI restriction activity using protoplast extracts, which allowed clear detection of CpaAI activity. Optimal digestion occurred between 2-4 hours incubation at 37° C. and produced a restriction pattern identical to that of BstUI, a commercial isoschizomer of CpaAI (FIG. 1A). Since all known BstUI isoschizomers catalogued in REBASE (Roberts, et al., 2010) are sensitive to methylation of both external cytosine residues within the 5′-CGCG-3′ recognition sequence, we next assessed the effect of external cytosine methylation by expression of the M.FnuDII methyltransferase (with 5′-m5CGCG-3′ methylation site of both DNA strands) from plasmid pFnuDIIMKn. M.FnuDII methylation protected pMTL85141, an E. coli-Clostridium shuttle vector (Heap, et al., 2009), from degradation by CpaAI and BstUI (FIG. 1B). While unmethylated substrates were significantly restricted after 2 hours incubation at 37° C., M.FnuDII-methylated substrates were completely resistant to cleavage, even after 8 h. Note that methylated pMTL85141 plasmid preparations, which also contains the pFnuDIIMKn methylating plasmid, migrated at a different molecular weight than unmethylated plasmid preparations. However, when we linearized the double-plasmid preparation, in addition to preparations of the two individual plasmids, we observed no detectable changes in plasmid size or unexpected products (FIG. 1B, right panel). In vitro methylation with commercial M.SssI (5′-m5CG-3′ methylation site) and M.CviPI (5′-Gm5C-3′ methylation site) methyltransferases also protected plasmids from digestion by CpaAI in protoplast extracts and commercial BstUI (not shown). Importantly, and unexpectedly, as elaborated below, whereas plasmid protection by the three methylases, M.FnuDII, M.SssI, and M.CviPI each conferred protection against CpaAI and BstUI digestion in vitro, methylation by the three enzymes were not equivalent in conferring the ability to transform C. pasteurianum with methylation-protected DNA.

Initial Electrotransformation of C. pasteurianum

To electrotransform C. pasteurianum, we employed a series of E. coli-Clostridium shuttle vectors which differ only in their Gram-positive origins of replication: pMTL82151 (pBP1 on from C. botulinum); pMTL83151 (pCB102 on from C. butyricum); pMTL84151 (pCD6 on from C. difficile); and pMTL85141 (pIM13 on from Bacillus subtilis) (Heap, et al., 2009).

We utilized conditions common to clostridial electrotransformation procedures (Table 2) and M.FnuDII-methylated DNA. Of the four vectors tested, pMTL83151, pMTL84151, and pMTL85141 yielded colonies using thiamphenicol selection, corresponding to electrotransformation efficiencies of 0.7×10¹, 0.3×10¹, and 2.4×10¹ transformants μg⁻¹ DNA, respectively. Accordingly, pMTL85141 was selected as the vector used for all subsequent electrotransformation work. Importantly, no transformants were obtained with unmethylated plasmid, validating the necessity to protect transforming DNA against the endogenous CpaAI restriction endonuclease. Interestingly, while in vivo methylation was essential for transformation, we did not obtain transformants when pMTL85141 was methylated in vitro with M.SssI or M.CviPI methyltransferases, although both enzymes protect pMTL85141 from digestion by CpaAI. This result is unexpected, and reinforces the degree of uncertainty and lack of obviousness of the choice of methylase which ultimately conferred successful C. pasteurianum transformation.

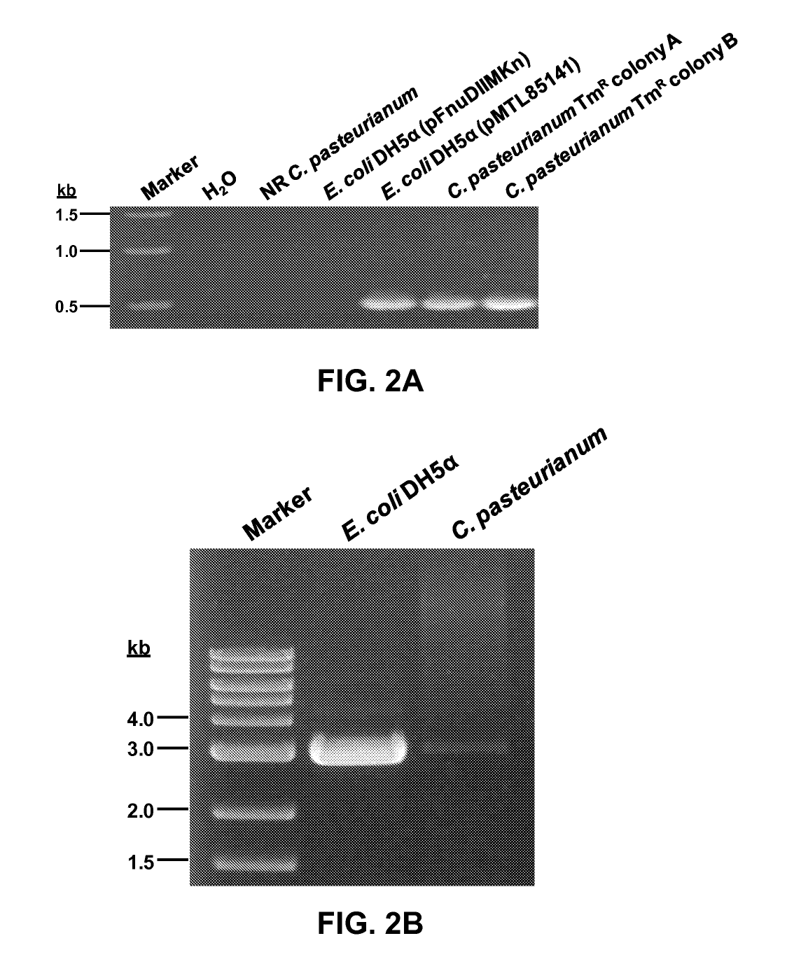

To confirm the presence of pMTL85141 in transformed colonies, we screened thiamphenicol-resistant colonies for the presence of the catP resistance marker within pMTL85141 using colony PCR (FIG. 2A). All of the colonies screened generated a single expected product of 518 bp. To further confirm the presence of plasmid and determine if rearrangements or recombinations occurred upon transfer to C. pasteurianum, plasmid pMTL85141 was isolated and purified from thiamphenicol-resistant colonies and digested with XhoI. XhoI digestion of all plasmid preparations from C. pasteurianum yielded a single band on a 1.0% agarose gel, similar to the digestion of pMTL85141 prepared from E. coli DH5α (FIG. 2B). The presence of the methyltransferase vector, pFnuDIIMKn, could not be detected in C. pasteurianum plasmid preparations.

2 Overcoming the Low Electroporation Transformation Efficiency of C. pasteurianum

The transformation efficiency obtained with electroporation of M.FnuDII-methylated pMTL85141 plasmid was 2.4×10 colonies per ug of DNA. This is a low transformation efficiency compared to efficiencies of up to 10⁶ transformants per ug DNA obtained in other Clostridia. Such a low transformation efficiency would be problematic for applying some genetic engineering technology in C. pasteurianum, such as intron-mediated gene knockouts and homologous recombination-based gene editing, which often require the availability of abundant colonies for screening due to their low success rates. Therefore, we set out to develop a protocol which enabled high efficiency transformation. We systematically evaluated the effect on transformation efficiency of changing a number of parameters, which were based on modifying the integrity of the C. pasteurianum cell wall to permit easier entry of foreign DNA into the cell, and optimizing the electroporation conditions. These investigations are detailed below and together with the Examples, would enable one skilled in the art to transform C. pasteurianum at high efficiency.

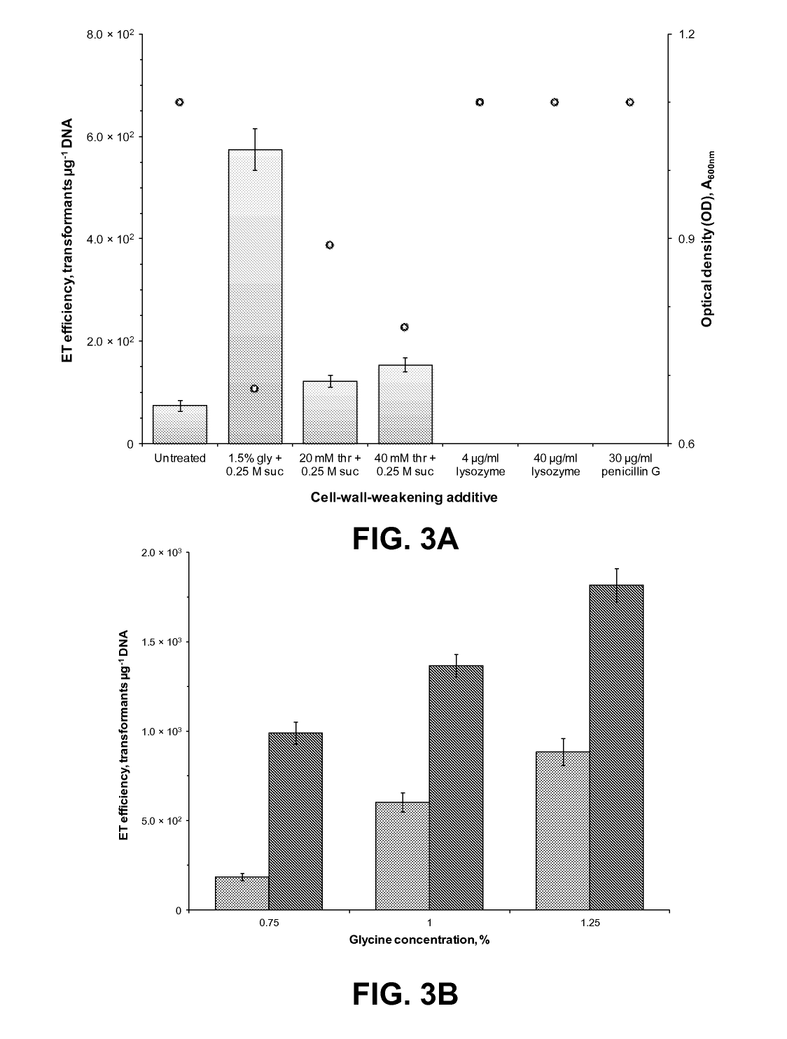

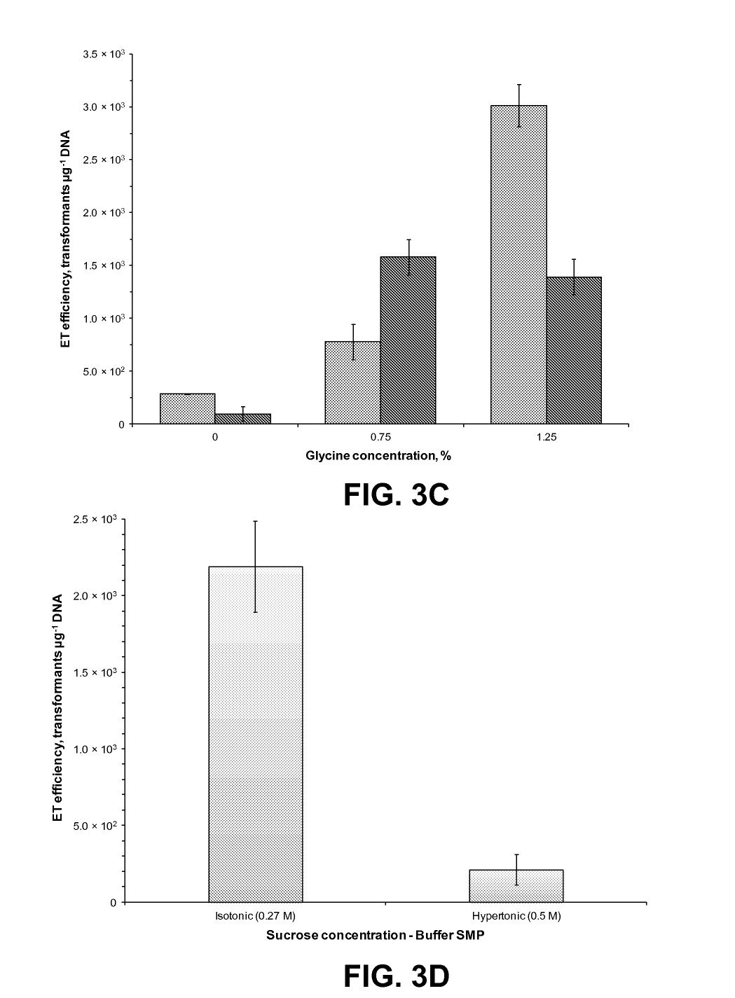

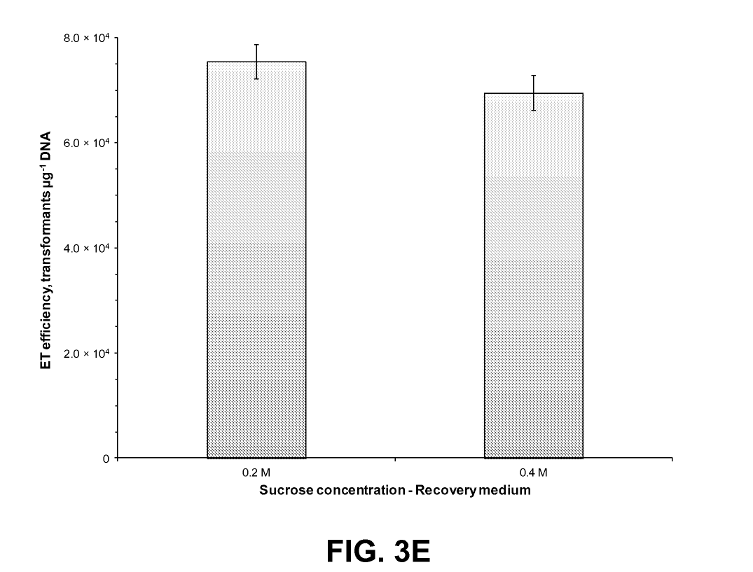

(i) Cell-wall-weakening. We first investigated the use of cell-wall-weakening agents due to their potential to greatly enhance electrotransformation by weakening of the Gram-positive cell wall. A screening experiment was conducted to identify potential additives capable of enhancing electrotransformation of C. pasteurianum, including glycine, DL-threonine, lysozyme, and penicillin G (FIG. 3A). Individually, we screened the effect of glycine and DL-threonine by supplying the additives in the presence of 0.25 M sucrose at the first signs of growth (OD₆₀₀ of 0.3-0.4) because cultures failed to grow to sufficient cell densities if glycine or DL-threonine were present without sucrose supplementation or if the additives were present at the time of inoculation. Cell growth rate was slightly reduced in the presence of both glycine and DL-threonine. On the other hand, lysozyme and penicillin G were screened by addition at the wash stage in the wash and electroporation buffer, followed by incubation at 37° C. for 30 minutes prior to electroporation. Additive concentrations were chosen based on previous electrotransformation studies with various species of Gram-positive bacteria. Of the four additives screened, only glycine and DL-threonine improved the electrotransformation efficiency. The samples treated with 40 μg/ml lysozyme and 30 μg/ml penicillin G even failed to grow during the outgrowth period following electroporation, potentially due to cell lysis. Despite a slight inhibition on cell growth, more than 7-fold enhancement of electrotransformation efficiency was attained using 1.5% glycine, compared to the control experiment with no cell-wall-weakening agent. Supplementation of 20 and 40 mM DL-threonine provided approximately 1.6- and 2.1-fold increases, respectively, in electrotransformation efficiency. Although glycine and DL-threonine have different mechanisms of cell wall disruption, combining glycine and DL-threonine treatments did not lead to a synergistic increase in electrotransformation efficiency.

As a result of the clear benefit of glycine on the electrotransformation efficiency, we set out to determine the optimum glycine regimen with respect to concentration and duration of exposure. This investigation was done concomitant with investigating the effect of sucrose on electrotransformation efficiency by providing osmoprotection during the various cell-wall-weakening glycine treatments. We tested glycine at 0.75, 1.0, and 1.25% in the presence of either 0.25 or 0.4 M sucrose, corresponding to nearly isotonic and hypertonic extracellular environments, respectively. The highest glycine concentration was selected as 1.25% to minimize growth inhibition, which becomes significant at concentrations equal to or greater than 1.5%. Increasing the sucrose concentration from 0.25 to 0.4 M led to a significant increase in electrotransformation efficiency under all glycine concentrations tested (FIG. 3B). To examine the effect of the duration of glycine exposure on electrotransformation efficiency, cultures were incubated with 0, 0.75, or 1.25% glycine in the presence of 0.4 M sucrose starting at an OD₆₀₀ of 0.4 for either 2.5 or 4.5 hours prior to washing and pulse delivery (FIG. 3C). Maximum electrotransformation efficiency was attained by exposing cells to 1.25% glycine for 2.5 hours in the presence of 0.4 M sucrose, a 10.7-fold increase compared to the untreated control culture. Interestingly, lower glycine concentrations could be compensated for by increasing the duration of exposure. When using a glycine concentration of 0.75% in the growth medium, 4.5 hours rather than 2.5 hours of exposure generated a greater electrotransformation efficiency at this lower glycine concentration, although the absolute gain in electrotransformation efficiency was still lower than with 1.25% glycine.

(ii) Osmoprotection. We continued to investigate the effect of the osmoprotectant concentration on electrotransformation efficiency during the subsequent washing and electroporation phase and the outgrowth phase following electroporation. Cells grown in the presence of 1.25% glycine and 0.4 M sucrose were washed and electroporated in the common clostridial SMP buffer containing either 0.27 M (isotonic) or 0.5 M (hypertonic) sucrose (FIG. 3D). SMP buffer outperformed other buffers tested, such as 10% PEG 8000, 15% glycerol, protoplast buffer with lysozyme omitted, and SMP buffer supplemented with 15% glycerol (data not shown). Hypertonic sucrose, which improved the electrotransformation efficiency when included during the growth phase, reduced electrotransformation efficiency by a factor of 10.3 when included at the washing and electroporation phase. Thus, 0.27 M sucrose was adopted as the optimum sucrose concentration in the wash and electroporation buffer.

To assess the effect of sucrose osmoprotection during cell recovery immediately following delivery of the electric pulse, cells were grown, made electrocompetent, pulsed, and resuspended in 10 ml 2xYTG containing either 0.2 or 0.4 M sucrose (FIG. 3E). Similar to the washing and electroporation phase, hypertonic sucrose again reduced electrotransformation efficiency, although the effect was modest (a 1.1-fold decrease), and thus, 0.2 M was adopted as the optimum sucrose concentration in the outgrowth medium.

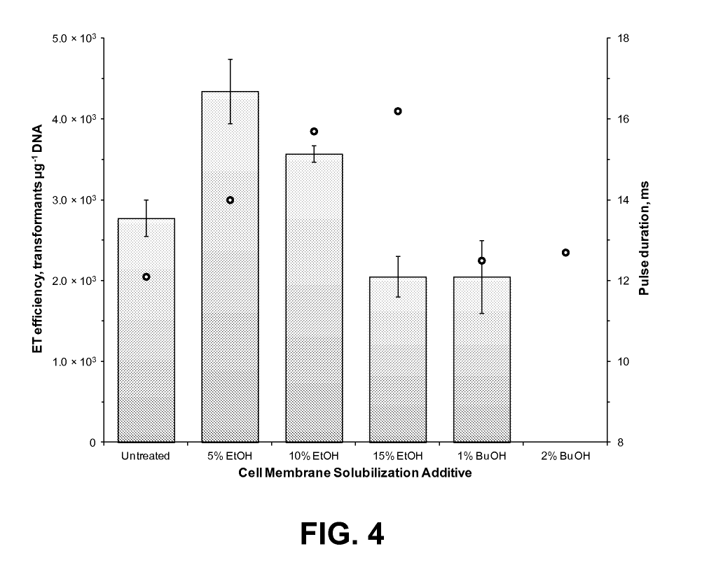

Cell membrane solubilization. After developing a regimen to weaken the exterior cell wall while supporting cell viability with sucrose osmoprotection, we next sought to enhance transfer of plasmid DNA to C. pasteurianum with the use of ethanol to solubilize the cell membrane, a strategy which has proved effective with some species of Gram-negative bacteria. We also extended this approach to butanol, which elicits a more pronounced toxic effect on cells. To achieve maximum membrane solubilization without adversely affecting cell viability, we utilized concentrations near the toxicity threshold for many species of Clostridium, which were up to 15% (v/v) for ethanol and 2% (v/v) for butanol. Five minutes prior to electroporation, ethanol or butanol was added directly to the cell-DNA suspension. Ethanol added at 5 and 10% provided a 1.6- and 1.3-fold respective increase in electrotransformation efficiency, compared to the control experiment with no ethanol treatment (FIG. 4). Butanol, and ethanol at an elevated concentration of 15%, proved to be detrimental to electrotransformation. The 2% butanol sample grew extremely slowly during the outgrowth period following electroporation. The addition of ethanol increased the pulse time constant, which may have influenced electrotransformation efficiency (FIG. 4). Butanol did not significantly affect the pulse time constant.

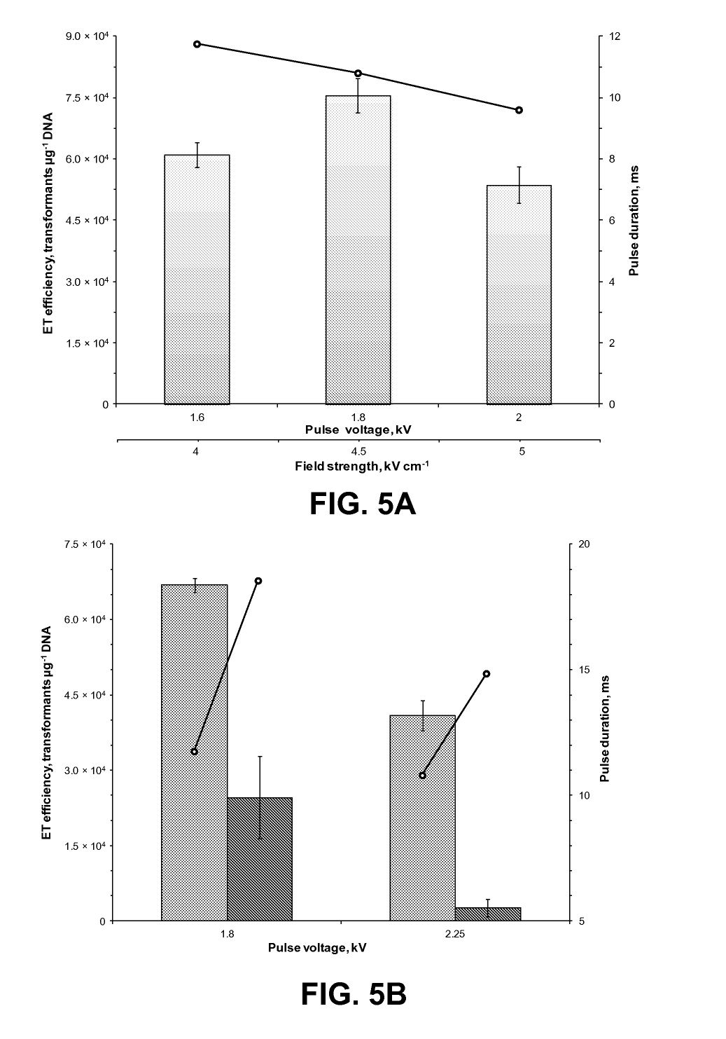

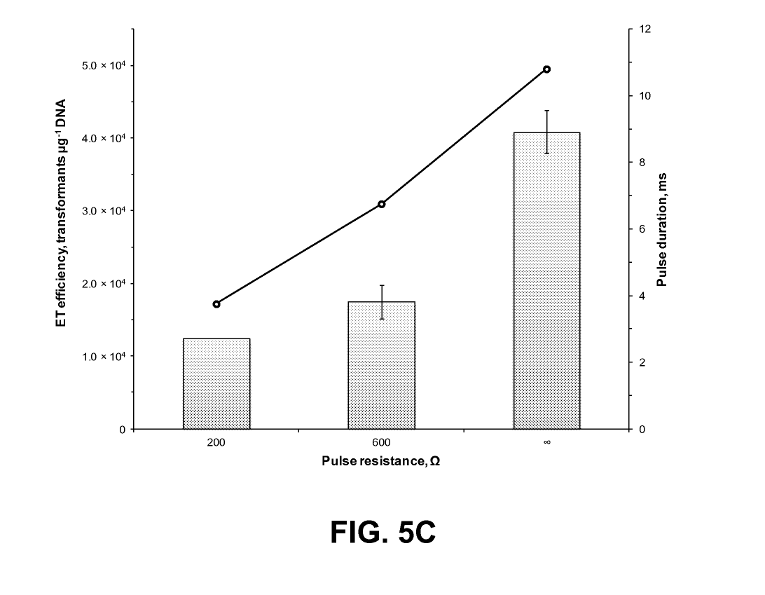

Electric pulse parameters. We investigated the effects of the electrical pulse with respect to voltage (i.e., field strength), capacitance, and resistance (FIG. 5A-C). In an initial screening experiment, low voltages in the range of 1.8-2.0 kV generated significantly more transformants than voltages of 2.0-2.5 kV (data not shown), which are representative of most electrotransformation protocols using species of Clostridium (Zhu, et al., 2005, Allen and Blaschek, 1988, Nakotte, et al., 1998, Zhou and Johnson, 1993). Hence, pulses of 1.6, 1.8, and 2.0 kV were administered, corresponding to field strengths of 4.0, 4.5, and 5.0 kV cm⁻¹ (FIG. 5A), using a capacitance of 25 μF and a resistance of ∞Ω (i.e., without the use of Pulse Controller module). A voltage of 1.8 kV was found to produce the greatest electrotransformation efficiency, although pulses of 1.6 and 2.0 kV only slightly reduced the electrotransformation efficiency. Pulse duration decreased by approximately 1 ms when increasing pulse voltage from 1.6 to 1.8 kV and from 1.8 to 2.0 kV. Next, capacitances of 25 and 50 μF were assessed at voltages of 1.8 and 2.25 kV, and ∞Ω (FIG. 5B). At both voltages, increasing the capacitance from 25 to 50 μF reduced electrotransformation efficiency by a factor of 2.7 (1.8 kV) and 15.6 (2.25 kV), respectively. Similarly, decreasing resistance from ∞Ω to 200 and 600Ω, at 2.25 kV and 25 μF was unproductive and resulted in a 3.3- and 2.3-fold decrease in electrotransformation efficiency, respectively (FIG. 5C). Pulse duration changes were not predictive of the effects on electrotransformation efficiency, as increases in the time constant accompanying changes in capacitance and decreases in the time constant accompanying changes in resistance both correlated with decreased electrotransformation efficiency.

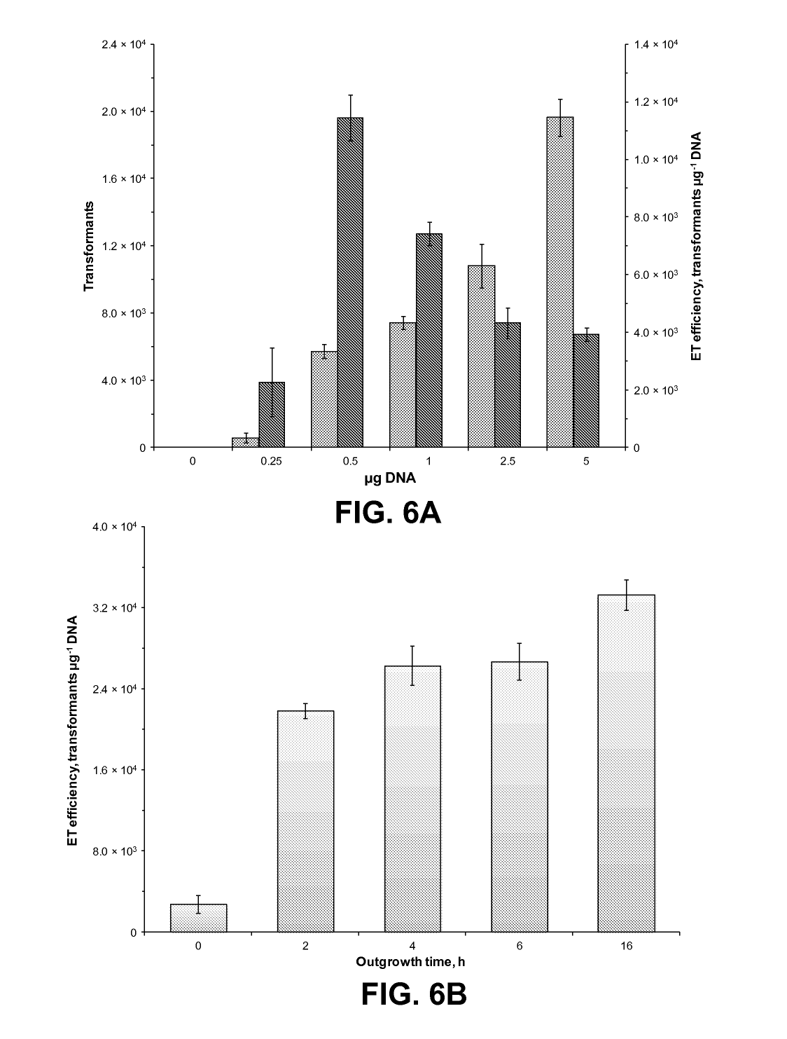

DNA quantity and outgrowth duration. Finally, we evaluated the effect of DNA amount on both number of transformants and electrotransformation efficiency (FIG. 6A) and the effect of the duration of outgrowth following electroporation. Although the total number of transformants was found to increase linearly between 0.5 and 5.0 μg of pMTL85141, the greatest electrotransformation efficiency occurred using 0.5 μg of plasmid DNA. Transformants could be detected at the lowest quantity of DNA tested, 0.25 μg, and saturation with pMTL85141 was not observed up to 5.0 μg, the highest quantity of DNA tested.

For assessing outgrowth duration, we incubated electroporated cells for 0, 2, 4, 6, or 16 hours prior to plating on selective medium. Growth in the form of gas formation and increased culture turbidity could be detected as early as 2 hours following transfer to recovery medium. Transformants could be obtained without recovery (i.e., 0 hours incubation), although at a significantly reduced efficiency (7.9- to 12.1-fold reduction compared to 2-16 hours incubation) (FIG. 6B). As expected, the greatest electrotransformation efficiency was attained using the longest recovery time tested (i.e., 16 hours), which was approximately 1.3-fold greater than at 4-6 hours outgrowth, during which time the electrotransformation efficiency was unchanged. While 16 hours of outgrowth is a convenient duration due to the length of the pre-growth and washing and electroporation phases, electrotransformation efficiency for clostridia is typically reported following 4-6 hours of outgrowth. Thus, the electrotransformation efficiencies reported here, all of which involved 16 hour outgrowth experiments, can be divided by 1.3 for comparison to other clostridial electroporation efficiencies.

### Application of the Electrotransformation Protocol to Other Vectors

Since many clostridial vectors favor the ermB determinant for erythromycin or clarithromycin selection, rather than catP-based thiamphenicol selection, we constructed pMTL85141ermB, a dual catP and ermB selectable plasmid. Comparable, high-level electrotransformation efficiencies (1.0-1.4×10⁴ transformants μg⁻¹ DNA) were obtained by selection of pMTL85141ermB using 15 μg/ml thiamphenicol, 4 μg/ml clarithromycin, or 20 μg/ml erythromycin. Control plasmid transformations lacking the ermB determinant failed to generate clarithromycin- or erythromycin-resistant colonies. Therefore, ermB-based clarithromycin or erythromycin selection is effective using C. pasteurianum.

To determine the generality of our high-efficiency electrotransformation protocol for other vectors, we also attempted electrotransfer of pSY6catP into C. pasteurianum. pSY6catP is a modified form of pSY6 (Shao, et al., 2007) whereby the ermB erythromycin-resistance determinant is replaced with catP from pMTL85141. pSY6 is one of several E. coli-Clostridium shuttle vectors (in addition to, e.g., the ClosTron system of vectors (Heap, et al., 2010)), which harbours the LI.ItrB group II intron machinery necessary for performing intron-mediated gene knockouts in clostridia. A pSY6-based vector was chosen because it possesses the same pIM13 replicon as pMTL85141, thereby eliminating potential variation in efficiency due to differences in the origin of replication. Unexpectedly, pSY6catP transformed C. pasteurianum at a significantly decreased efficiency of 1.1×10¹ transformants μg⁻¹ DNA, an efficiency approximately 1.000-fold lower than achieved with pMTL85141. To rule out a vector size effect on the reduction in electrotransformation efficiency (pSY6catP is 8,498 bp, whereas pMTL85141 is 2,963 bp), we also attempted to transform pHT3, a 7,377 bp vector with the same fundamental vector components as pMTL85141ermB, in addition to a heterologous lacZ gene from Thermoanaerobacterium thermosulfurogenes EM1 (Tummala, et al., 1999) (Table 1). Unlike pSY6catP, pHT3 transformed at a high efficiency of 1.8×10⁴ transformants μg⁻¹ DNA, which is comparable to pMTL85141ermB. Therefore, the dramatic reduction in electrotransformation efficiency is likely not due to differences in plasmid size. At this point, we hypothesize the presence of an additional unidentified restriction system which targets certain common site(s) of pSY6catP, but not pMTL85141, pMTL85141ermB, or pHT3, much like the dcm-methylation-dependent restriction systems recently addressed in C. thermocellum and C. ljungdahlii. Our observation of the transformability of in-vivo-methylated plasmids, but not in-vitro-methylated plasmids, may also be the result of an unidentified methylation-dependent restriction system, which may or may not be the same one affecting pSY6catP. Nonetheless, even with the reduced electrotransformation efficiency of pSY6catP, we have used it to successfully introduce type II introns into the C. pasteurianum genome in preliminary experiments.

The unexpected result that there are some vectors, such as pSY6catP, which, even with M.FnuDII methylation, still fail to transform C. pasteurianum at high efficiency, emphasizes the value and lack of obviousness of our discovery of vectors such as pMTL85141, pMTL85141ermB, pHT3 that are capable of transforming C. pasteurianum at high efficiency once they are methylated in vivo by M.FnuDII.

In summary, we developed for the first time a high-efficiency transformation protocol for C. pasteurianum. Many variables needed to be carefully tuned to achieve optimal transformation efficiency, and there were several unexpected findings during the process of creating the invention. First, we determined that methylation was required to protect transformed plasmids from degradation by C. pasteurianum's CpaAI restriction enzyme system. However, surprisingly, not all methyltransferases which blocked CpaAI digestion activity in vitro were useful for protecting plasmids for transformation into C. pasteurianum. Only in vivo methylation by growth in a restriction deficient strain of E. coli, such as ER1821, harbouring the M.FnuDII methylation gene on a plasmid (in our case on plasmid pFnuDIIMKn), successfully protected plasmid for transformation into C. pasteurianum. At this point, we do not know whether the failure of the methylases M.SssI and M.CviPI to support transformation of C. pasteurianum was due to the methylases themselves or the use of the mythylases for in vitro, rather than in vivo, methylation.

Next, we sought to weaken the cell wall to try to increase transformation efficiency. Again, there were some unexpected results. While glycine, DL-threonine, lysozyme, and penicillin G have all been used for weakening the cell wall of gram-positive bacteria, in our experimental conditions, only glycine and DL-threonine significantly enhanced the level of electroporation. Importantly, it was also important to optimize the timing and concentration of glycine exposure to C. pasteurianum. Exposing the C. pasteurianum cells too early in their growth cycle, or with too great a concentration of glycine, led to a major loss of cell viability. The optimum glycine regimen for C. pasteurianum involved exposure of early exponential phase cells (at OD₆₀₀ of 0.3-0.4) to 1.25% glycine for 2-3 hours. To our knowledge, this is the first use of glycine as a cell-wall weakening and electroporation-enhancing agent within the Clostridium genus.

As treatment with glycine compromised the cell wall, it became important to stabilize C. pasteurianum cells osmotically during the glycine treatment. However, here again, it was important to carefully monitor the time of application and concentration of the osmoprotectant agent. We discovered that the use of hypertonic 0.4 M sucrose in the growth medium significantly enhanced electrotransformation (FIG. 3B). However, adding hypertonic sucrose in the recovery medium following electroporation was not as effective as treatment with hypotonic 0.2 M sucrose.

We also optimized the strength and duration of the electric field applied during electroporation. Generally, tailoring of the electric pulse was interdependent on the various optimizations of cell-wall weakening treatment; changing one required changing the other to maximize electrotransformation efficiency. We discovered that in contrast to the normal voltages of 2.0-2.5 kV (or 5.0-6.25 kV cm⁻¹) which are used to transform other Clostridium bacteria, glycine-treated C. pasteurianum was found to benefit from a lower voltage of 1.8 kV (4.5 kV cm⁻¹; FIG. 5A). Applying a capacitance of 25 μF and ∞Ω to the 1.8 kV pulse was most beneficial.

Once the cell wall is weakened, the foreign DNA must still cross through the cell membrane. Ethanol is known as a membrane-solubilizing agent that enhances the transformation of Escherichia coli. However, it has not previously been used to improve electrotransformation of Clostridia. We discovered that adding 5-10% ethanol to the electroporation mixture had a clear positive effect on electrotransformation (FIG. 4).

Taken together, with the optimization of various electroporation parameters, and the careful timed additions of specific concentrations of cell wall and cell membrane modifying agents, we achieved a 3.000-fold increase in electroporation efficiency compared to our initial electrotransformation attempt using common clostridial electroporation conditions (Table 3). Our final maximum transformation efficiency was 7.5×10⁴ tranformants ug⁻¹ DNA, which is among the highest reported in the Clostridium genus. This transformation efficiency is sufficiently high to perform all of the normal genetic engineering manipulations needed to produce high-producing industrial strains.

Finally, in our invention, we disclosed for the first time, a number of vectors and selection markers, which are effective for transforming C. pasteurianum. These new tools included vectors with the pCB102 origin of replication from C. butyricum (pMTL83151), pCD6 origin of replication from C. difficile (pMTL84151), and the pIM13 from B. subtilis (pMTL85141, pMTL85141ermB, and pHT3) and vectors carrying catP and ermB genes for thiamphenicol and erythromycin/clarithromycin selection, respectively. We established that concentrations of 10-15 μg/ml thiamphenicol, 4 μg/ml clarithromycin, and 20 μg/ml erythromycin were appropriate for selection of transformed colonies. Importantly, not all vectors that we tried to transform were equally effective in generating transformation colonies in C. pasteurianum, in spite of being adequately protected from CpaAI degradation by methylation. In particular, the class of pSY6 vectors, which are conventionally used to transform group II intron-mediated gene knockout machinery into clostridial cells (Shao, et al., 2007), did not transform efficiently with our protocol. This implies that the vectors that we identified as supporting high-efficiency transformation were not obvious since we could not predict beforehand which vectors would and would not transform C. pasteurianum efficiently.

Nonetheless, even with the pSY6 vectors that transformed C. pasteurianum poorly, we were able to generate sufficient colonies following transformation to detect successful gene knockout events.

Below, further examples are provided of the detailed use of the invention to achieve successful transformation of C. pasteurianum.

### EXAMPLES

The following examples are provided by way of illustration and not by limitation.

### Example 1

The bacterial strains, plasmids, and oligonucleotides utilized in this invention are listed in Table 1. E. coli DH5α was utilized for routine vector construction and propagation, and E. coli ER1821 for maintenance of M.FnuDII-methylated E. coli-C. pasteurianum shuttle vectors. C. pasteurianum ATCC™ 6013 (Winogradsky 5; W5) was acquired from the American Type Culture Collection (Manassas, Va., USA). Modular pMTL-series shuttle vectors (Heap, et al., 2009) were kindly provided by Prof. Nigel Minton (University of Nottingham, Nottingham, UK). Plasmids pFnuDIIM (Lunnen, et al., 1988), pSC12 (Zhao, et al., 2003), and pSY6 (Shao, et al., 2007) were respectively provided by Dr. Geoffrey Wilson (New England Biolabs, Inc. (NEB), Ipswich, Mass., USA), Prof. George Bennett (Rice University, Houston, Tex., USA), and Prof. Sheng Yang (Shanghai Institutes for Biological Sciences, Shanghai, China). Plasmids pHT3 (Tummala, et al., 1999) and pIMP1 (Mermelstein, et al., 1992) were provided by Prof. Terry Papoutsakis (University of Delaware, Newark, Del., USA). Oligonucleotide primers were synthesized and purified by Integrated DNA Technologies (IDT; Iowa City, Iowa, USA) using standard desalting.

### Bacteria Growth and Maintenance

Unless stated otherwise, all chemicals were purchased from Sigma-Aldrich (St. Louis, Mo., USA) and stock solutions were prepared according to the manufacturer's recommendations. E. coli strains were grown aerobically at 37° C. in lysogeny broth (LB; 10 g/l NaCl, 5 g/l Bacto yeast extract, and 10 g/l Bacto tryptone). Solid and liquid cultures of recombinant E. coli were supplemented with 100, 34, or 30 μg/ml of ampicillin, chloramphenicol, and kanamycin, respectively. For selection of strains harboring two compatible plasmids, antibiotic concentrations were reduced by 50%. Recombinant E. coli stocks were stored at −80° C. in 15% glycerol. Unless specified otherwise, growth and manipulation of C. pasteurianum was performed in a controlled anaerobic atmosphere (85% N₂, 10% H₂, and 5% CO₂) within an anaerobic chamber (Plas-Labs, Inc.; Lansing, Mich., USA). Oxygen was purged from growth medium by autoclaving and trace O₂ was reduced using a palladium catalyst fixed to the heating unit of the anaerobic chamber. Agar-solidified medium was prepared aerobically and allowed to equilibrate within the anaerobic chamber for at least 36 hours prior to use. Anaerobic conditions were monitored by addition of 1 mg/l resazurin to both solid and liquid media. Solid and liquid cultures of recombinant C. pasteurianum were supplemented with 15 μg/ml thiamphenicol. Cells were maintained as spores on solidified 2xYTG (16 g/l Bacto tryptone, 10 g/l Bacto yeast extract, 5 g/l glucose, 5 g/l NaCl, and 12 g/l agar) plates. Sporulated agar plate stocks were prepared by streaking colonies from an exponential-phase culture (OD₆₀₀ of 0.4-0.6) and cultivating for more than seven days under anaerobic conditions, followed by exposure and storage in air at 4° C. for up to two months. For long-term storage, vegetative stock cultures (OD₆₀₀ of 0.4-0.6) were prepared and stored at −80° C. in 10% glycerol by inoculating a single sporulated plate colony into 10 ml 2xYTG and heat shocking at 80° C. for 10 minutes to induce germination.

### DNA Isolation and Manipulation

Plasmid DNA was extracted and purified from E. coli DH5α and ER1821 using an EZ-10 Spin Column Plasmid DNA Miniprep Kit from Bio Basic, Inc. (Markham, ON, Canada). Recombinant DNA manipulations were performed according to standard procedures (Sambrook, et al., 1989). Taq DNA polymerase, restriction endonucleases, CpG (M.SssI) and GpC (M.CviPI) methyltransferases, Quick Ligation Kit, and 1 kb DNA ladder were purchased from NEB (Ipswich, Mass., USA). Pfu DNA polymerase and RNase A were purchased from Bio Basic, Inc. (Markham, ON, Canada). All commercial enzymes and kits were used according to the manufacturer's instructions.

Colony PCR of wild-type and recombinant C. pasteurianum was performed by suspending single colonies in 50 μl colony lysis buffer (20 mM Tris-HCl, pH 8.0, containing 2 mM EDTA and 1% Triton X-100), heating in a microwave for 2 minutes at maximum power setting, and adding 1 μl of the resulting cell suspension to a 9 μl PCR containing Standard Taq DNA Polymerase (NEB; Ipswich, Mass., USA). An initial denaturation of 5 minutes at 95° C. was employed to further cell lysis. Colonies screened in this manner by suspension in deionized H₂O failed to yield appreciable amplification.

Vector construction

Plasmid pFnuDIIMKn was derived from pFnuDIIM to allow methylation of E. coli-C. pasteurianum shuttle vectors and possesses a kanamycin-resistance determinant, as both pFnuDIIM (Lunnen, et al., 1988) and the E. coli-C. pasteurianum shuttle vectors used in this study carry the same chloramphenicol-resistance marker. First, an FRT-kan-FRT PCR cassette was amplified from plasmid pKD4 (Datsenko and Wanner, 2000) using primers KnFRT.BlpI.S (SEQ ID NO: 1) and KnFRT.XhoI.AS (SEQ ID NO: 2) and inserted into the MCS of BlpI/XhoI-digested pET-20b(+) (Novagen; Madison, Wis., USA) to generate pETKnFRT. Next, the FRT-kan-FRT cassette was digested out of pETKnFRT using ScaI and EcoRI and subcloned into the corresponding restriction sites within the catP gene of pFnuDIIM to yield pFnuDIIMKn.

Plasmid pSY6catP was derived from pSY6 (Shao, et al., 2007) by swapping the ermB marker with the catP determinant from pSC12 (Zhao, et al., 2003). The internal BsrGI recognition site within the coding sequence of catP was mutated by introducing two silent mutations using splicing by overlap extension (SOE) PCR to prevent interference with future group II intron retargeting, which requires use of BsrGI. The catP gene was amplified in two parts from template pSC12 using primer sets catP.BcII.S (SEQ ID NO: 5)/pSC12.SOE.AS (SEQ ID NO: 6) and pSC12.SOE.S (SEQ ID NO: 7)/catP.ClaI.AS (SEQ ID NO: 8) with 22 bp of overlap between products. The resulting overlapping PCR products were separated on a 2.0% agarose gel, pierced three times with a P10 micropipette tip, and used as template in a SOE PCR by cycling for 10 cycles prior to adding primers catP.BcII.S (SEQ ID NO: 5) and catP.ClaI.AS (SEQ ID NO: 8) and cycling for 25 additional cycles. The mutated PCR product was purified using a EZ-10 Spin Column PCR Products Purification Kit (Bio Basic, Markham, ON, Canada), digested with BclI/ClaI, and inserted into the corresponding sites of pSY6 to generate pSY6catP.

Plasmid pMTL85141ermB was derived from pMTL85141 via insertion of the ermB marker from pIMP1 into pMTL85141. The ermB gene and associated promoter was PCR-amplified from template pIMP1 using primers ermB.NdeI.S (SEQ ID NO: 3) and ermB.Pvul.AS (SEQ ID NO: 4). The resulting 1,238 bp PCR product was purified using an EZ-10 Spin Column PCR Products Purification Kit (Bio Basic, Markham, ON, Canada), digested with NdeI/PvuI, and inserted into the corresponding sites of pMTL85141 to generate pMTL85141ermB.

### Preparation of Electrocompetent Cells and Electrotransformation

For preparation of electrocompetent cells of C. pasteurianum using the high-level protocol, a seed culture was first prepared by inoculating 20 ml of reduced 2xYTG with 0.2 ml of a thawed glycerol stock. The culture was then 20⁻²-diluted and, following overnight growth at 37° C., 1 ml of the seed culture was transferred to a 125 ml Erlenmeyer flask containing 20 ml of reduced 2xYTG. Cells were grown to early exponential phase (OD₆₀₀ of 0.3-0.4), at which time filter-sterilized stock solutions of 2 M sucrose and 18.77% glycine were added to respective concentrations of 0.4 M and 1.25%. Growth was resumed until the culture attained an OD₆₀₀ of 0.6-0.8 (approximately 2-3 h) and 20 ml culture was transferred to a 50 ml pre-chilled, screw-cap centrifuge tube. At this point, all manipulations were performed at 4° C. using an ice-bath and pre-chilled reagents. Cells were removed from the anaerobic chamber and collected by centrifugation at 8,500×g and 4° C. for 20 minutes. The resulting cell pellet was returned to the anaerobic chamber and washed once in 5 ml of filter-sterilized SMP buffer (270 mM sucrose, 1 mM MgCl₂, and 5 mM sodium phosphate, pH 6.5). Following centrifugation, the final cell pellet was resuspended in 0.6 ml SMP buffer.

For transfer of plasmids to C. pasteurianum, E. coli-C. pasteurianum shuttle vectors were first co-transformed with pFnuDIIMKn into E. coli ER1821 to methylate the external cytosine residue within 5′-CGCG-3′ tetranucleotide recognition sites of CpaAI. Plasmid mixtures were then isolated and 0.5 μg, suspended in 20 μl of 2 mM Tris-HCl, pH 8.0, was added to 580 μl of C. pasteurianum electrocompetent cells. The cell-DNA mixture was transferred to a pre-chilled electroporation cuvette with 0.4 cm gap (Bio-Rad; Richmond, Calif., USA), 30 μl of cold 96% ethanol was added, and the suspension was incubated on ice for 5 minutes. A single exponential decay pulse was applied using a Gene Pulser (Bio-Rad, Richmond, Calif., USA) set at 1.8 kV, 25 μF, and ∞Ω, generating a time constant of 12-14 ms. Immediately following pulse delivery, the cuvette was flooded with 1 ml 2xYTG medium containing 0.2 M sucrose and the entire suspension was transferred to 9 ml of the same medium. Recovery cultures were incubated for 4-6 hours prior to plating 50-250 μl aliquots onto 2xYTG agar plates containing 15 μg/ml thiamphenicol, 4 μg/ml clarithromycin, or 20 μg/ml erythromycin. Plates were incubated for 2-4 days under secondary containment within 3.4 L Anaerobic Jars each equipped with a 3.5 L Anaerobic Gas Generating sachet (Oxoid Thermo Fisher; Nepean, ON, Canada).

We claim:

1. A method for introducing recombinant DNA constructs into one or more bacterium.

2. The method of claim 1 wherein said bacterium is a gram-positive bacteria.

3. The method of claim 1 wherein said bacterium belongs to the genus Clostridia.

4. The method of claim 1 wherein said bacterium is Clostridium pasteurianum.

5. The method of claim 1 wherein said method involves the delivery of one or more electrical pulses to said bacterium.

6. The method of claim 1 wherein said method involves the use of methylation to block the activity of a restriction enzyme within said bacterium.

7. The method of claim 1 wherein said method involves the use of a methylase which methylates a cytosine residue within the deoxyribonucleotide sequence 5′-cytosine-guanine-cytosine-guanine-3′.

8. The method of claim 1 wherein said method involves the use of a cell-wall weakening agent.

9. The method of claim 8 wherein said cell-wall weakening agent is selected from the group consisting of glycine and DL-threonine.

10. The method of claim 9 wherein said method involves the use of an osmoprotectant agent.

11. The method of claim 10 wherein said osmoprotectant is selected from the group consisting of sucrose, lactose, sorbitol, and mannitol.

12. The method of claim 9 where said method involves the use of ethanol.

13. The method of claim 4 where said recombinant DNA construct contains an origin of replication selected from the group consisting of pCB102 from Clostridium butyricum, pCD6 from Clostridium difficile, and pIM13 from Bacillus subtilis.

14. The method of claim 4 wherein said recombinant DNA construct contains a DNA sequence which encodes an enzyme that confers resistance to an antibiotic selected from the group consisting of thiamphenicol, clarithromycin, and erythromycin.

15. The method of claim 4 wherein said method comprises:
(i) the delivery of one or more electrical pulses to said bacterium.
(ii) the use of one or more cell-wall weakening agents selected from the group comprising glycine and DL-threonine.
(iii) the use of one or more osmoprotectants selected from the group consisting of sucrose, lactose, mannitol, and sorbitol.

16. A bacterial cell which contains one or more recombinant DNA constructs.

17. The bacterial cell of claim 16 wherein said bacterial cell is from a gram-positive bacteria.

18. The bacterial cell of claim 16 wherein said bacterial cell is from a bacteria that is a member of the genus Clostridium.

19. The bacteria cell of claim 16 wherein said bacterial cell is from Clostridium pasteurianum.

20. The bacterial cell of claim 16 wherein said one or more recombinant DNA constructs were introduced into said bacterial cell by a method involving the delivery of one or more electrical pulses to said bacterium.

21. The bacterial cell of claim 16 wherein said one or more recombinant DNA constructs were introduced into said bacterial cell by a method involving the use of methylation to block the activity of a restriction enzyme within said bacterium.

22. The bacterial cell of claim 16 wherein said one or more recombinant DNA constructs were introduced into said bacterial cell by a method involving the use of a methylase which methylates a cytosine residue within the deoxyribonucleotide sequence 5′-cytosine-guanine-cytosine-guanine-3′.

23. The bacterial cell of claim 16 wherein said one or more recombinant DNA constructs were introduced into said bacterial cell by a method involving the use of a cell-wall weakening agent.

24. The bacterial cell of claim 23 wherein said cell-wall weakening agent is selected from the group consisting of glycine and DL-threonine.

25. The bacterial cell of claim 24 wherein said method involves the use of an osmoprotectant agent.

26. The bacterial cell of claim 25 wherein said osmoprotectant is selected from the group consisting of sucrose, lactose, sorbitol, and mannitol.

27. The bacterial cell of claim 24 where said method involves the use of ethanol.

28. The bacterial cell of claim 19 where said one or more recombinant DNA constructs contain an origin of replication selected from the group consisting of pCB102 from Clostridium butyricum, pCD6 from Clostridium difficile, and pIM13 from Bacillus subtilis.

29. The bacterial cell of claim 19 wherein said one or more recombinant DNA constructs contain a DNA sequence which encodes an enzyme that confers resistance to an antibiotic selected from the group consisting of thiamphenicol, clarithromycin, and erythromycin.

30. The bacterial cell of claim 19 wherein said one or more recombinant DNA constructs are introduced into said bacterial cell by a method comprising:
(i) the delivery of one or more electrical pulses to said bacterium.
(ii) the use of one or more cell-wall weakening agents selected from the group comprising glycine and DL-threonine.
(iii) the use of one or more osmoprotectants selected from the group consisting of sucrose, lactose, mannitol, and sorbitol.
